(19) United States
(12) Patent Application Publication
(10) Pub. No.: US 2011/0212160 A1
(43) Pub. Date: Sep. 1, 2011

(54) CONFIGURABLE MICROSCOPIC MEDICAL PAYLOAD DELIVERY DEVICE TO DELIVER NUCLEAR SIGNALING PROTEINS TO SPECIFIC CELLS TO MANAGE DIABETES MELLITUS AND GENETIC DEFICIENCY DISORDERS

(76) Inventors: LANE BERNARD SCHEIBER, Annandale, VA (US); Lane Bernard Scheiber, II, Grosse Ile, MI (US)

(21) Appl. No.: 12/715,377

(22) Filed: Mar. 1, 2010

(51) Int. Cl. A61K 38/16; A61K 9/10; C07K 14/00; A61K 9/127; A61P 43/00

(52) U.S. Cl. 424/450; 424/484; 530/350; 530/395; 514/1.2; 514/20.6

(57) ABSTRACT

The innovative treatment strategy described here utilizes configurable microscopic medical payload delivery devices to act as a transport vector to deliver nuclear signaling proteins to specific cell types in the body. Utilizing probes on the exterior of the transport device, transport device locate specific target cell types in the body. Once a specific target cell type has been encountered, the configurable microscopic medical payload delivery device inserts its payload of nuclear signaling proteins into the target cell type. By delivering nuclear signaling proteins to specific cell types, genes can be activated or inactivated in those specific cell types. These medically therapeutic nuclear signaling proteins are intended to improve cell function or the longevity of the cell or eliminate cells that pose a hazard to the general health of the body.

### CROSS-REFERENCE TO RELATED APPLICATIONS

None.

### STATEMENT REGARDING SPONSORED RESEARCH OR DEVELOPMENT

None.

### REFERENCE TO SEQUENCE LISTING, A TABLE, OR COMPUTER LISTING COMPACT DISC APPENDIX

Not applicable.

©2010 Lane B. Scheiber and Lane B. Scheiber II. A portion of the disclosure of this patent document contains material which is subject to copyright protection. The copyright owners have no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device intended to correct a protein deficiency or genetic deficiency in the body by utilizing a configurable microscopic medical payload delivery device to insert one or more nuclear signaling proteins into one or more specific cell types in the body to improve cell function.

2. Description of Background Art

For purposes of this text a ‘nuclear signaling protein’ molecule (NSP) is a protein molecule intended to attach to nuclear deoxyribonucleic acid, by means of zinc fingers, for the purpose of initiating or inhibiting the process of transcription of a segment of the deoxyribonucleic acid. Nuclear signaling protein molecules include nuclear receptors (NR), nuclear binding proteins (NBP), and artificial transcription factors (ATF).

Proteins are comprised of one or more linear strings of amino acids. Particular segments of a protein may be termed a ‘domain’. Zinc fingers refer to small protein domains that are folded, with these folds stabilized by one or more zinc ions. The physical structure of the folding of the zinc finger DNA binding domain facilitates the protein molecule's ability to attach to deoxyribonucleic acid (DNA), ribonucleic acid (RNA), other proteins or small molecules.

In the case whereby a zinc finger DNA binding domain facilitates a nuclear binding protein molecule to bind to DNA, the physical structure of the folding of the zinc finger DNA binding domain engages and makes contact with a specific sequence of DNA bases. A particular zinc finger DNA binding domain therefore attaches to a specific sequence of DNA bases. Once a zinc finger DNA binding domain binds to the DNA, this facilitates or represses the possible transcription of a specific segment of the DNA located near the site where the zinc finger DNA binding domain caused the nuclear binding protein molecule to bind to the DNA.

Nuclear receptors are proteins that sense the presence of ligands such as steroid, thyroid hormones, and certain other proteins. The description of a nuclear receptor is varied in the literature. For purposes of this text, nuclear receptors are commonly comprised of a N-terminal domain, one or more zinc finger DNA binding domains, one or more transactivation domains, a hinge region, a ligand binding domain and a C-terminal domain. In general, nuclear receptors exist in a nonactive form until a ligand binds to the ligand binding domain. Prior to a ligand binding to the nuclear receptor, the nuclear receptor may be prevented from attaching to DNA by the presence of neutralizing proteins. A ligand binding to the ligand binding domain causes: (1) a conformational change in the nuclear receptor which activates the nuclear receptor and (2) removal of the presence of any neutralizing proteins. Separating neutralizing proteins from a nuclear receptor frees the nuclear receptor to traverse to a specific site along the deoxyribonucleic acid. The zinc finger DNA binding domain attaches the nuclear receptor to the DNA at a specific site along the DNA. The transactivation domain interacts with other transcription proteins that ultimately assemble to form a transcription complex. The transcription complex transcribes genetic information in the DNA. The hinge region is thought to facilitate changes in the three-dimensional shape of the nuclear protein.

Nuclear receptors are subset into at least four categories. The four categories include: (1) Nuclear receptors that originally reside in the cytoplasm and sense the presence of extrinsic ligands, (2) Nuclear receptors that reside in the cytoplasm and sense intrinsic ligands, (3) Nuclear receptors that reside in the nucleus and sense the presence of extrinsic ligands, and (4) Nuclear receptors that reside in the nucleus and sense the presence of intrinsic ligands. Nuclear receptors that sense ‘intrinsic’ ligands are often referred to as ‘orphan’ nuclear receptors.

When an extrinsic ligand or an intrinsic ligand binds to a nuclear receptor that resides in the cytoplasm, the now activated nuclear receptor traverses the cytoplasm, enters the nucleus, traverses the nucleus and attaches to the DNA at a specific binding site as dictated by the zinc finger DNA binding domain present within the nuclear receptor.

When an extrinsic ligand or an intrinsic ligand binds to a nuclear receptor that resides in nucleus, the now activated nuclear receptor traverses the nucleus and attaches to the DNA at a specific binding site as dictated by the zinc finger DNA binding domain present within the nuclear receptor.

Examples of extrinsic ligands are steroids and thyroid hormones, which enter the cell from the external environment surrounding the cell. Once an extrinsic ligand enters the cell it attaches to the ligand binding domain of a nuclear receptor residing in the cytoplasm or it attaches to the ligand binding domain of a nuclear receptor residing in the nucleus. Upon an extrinsic ligand binding to the binding domain of a nuclear receptor the result is the nuclear receptor becomes activated, traverses to a specific binding site on the DNA as dictated by the zinc finger DNA binding domain present within the nuclear receptor and binds to that site on the DNA. The binding of the activated nuclear receptor to the DNA at a specific site along the DNA causes coalescing of transcription proteins that results in the assembly of a transcription complex. The transcription complex then transcribes a segment of DNA.

An intrinsic ligand refers to an activating molecule generated inside the cell that traverses the cytoplasm and binds to the ligand binding domain of a nuclear receptor. Upon an intrinsic ligand binding to the ligand binding domain of a nuclear receptor the result is the nuclear receptor becomes activated, traverses to a specific binding site on the DNA as dictated by the zinc finger DNA binding domain present within the nuclear receptor and binds to that site on the DNA. The binding of the activated nuclear receptor to the DNA at a specific site along the DNA causes coalescing of transcription proteins that results in the assembly of a transcription complex. The transcription complex then transcribes a segment of DNA.

Nuclear binding proteins (NBP) do not require the binding of a ligand and activation prior to physically binding to the DNA. Nuclear binding proteins are subset into at least two categories, which include: (1) immediately active nuclear binding proteins (iaNBP) and (2) delayed activity nuclear binding proteins (daNBP).

Immediately active nuclear binding proteins (iaNBP) at a minimum are comprised of a zinc finger DNA binding domain, a transactivation domain, a C-terminal region and a N-terminal region. There may exist more than one transactivation domains. There may exist a hinge domain. There may exist more than one zinc finger DNA binding domain. The zinc finger DNA binding domain attaches the nuclear receptor to the DNA at a specific site along the DNA. The transactivation domain interacts with transcription proteins that ultimately assemble to form a transcription complex. The iaNBP attaches to a specific segment of the DNA as dictated by the zinc finger DNA binding domain present within the iaNBP. Once the iaNBP has bound to the DNA, the iaNBP activates transcription proteins to assemble into a transcription complex. The transcription complex then transcribes a segment of the DNA.

Delayed activity nuclear binding proteins (daNBP) at a minimum are comprised of a ligand binding domain, a zinc finger DNA binding domain, a transactivation domain, a C-terminal region and a N-terminal region. There may exist more than one transactivation domains. There may exist a hinge domain. There may exist more than one zinc finger DNA binding domain. The zinc finger DNA binding domain attaches the nuclear receptor to the DNA at a specific site along the DNA. The transactivation domain interacts with transcription proteins that ultimately assemble to form a transcription complex. The ligand binding domain acts as a receptor for a ligand to bind to. The daNBP binds to the DNA per attachment of the zinc finger DNA binding domain to the DNA. The daNBP may sit attached to the DNA without activating transcription proteins, until a ligand becomes attached to the daNBP's ligand binding domain. Once a ligand binds to the ligand binding domain of the daNBP, a conformation change occurs in the daNBP molecule. The conformation change in the daNBP activates transcription proteins. Activated transcription proteins assemble into a transcription complex. The transcription complex then transcribes a segment of the DNA.

Artificial transcription factors (ATF) have been created that utilize zinc finger DNA binding domains to attach to DNA. Artificial transcription factors are comprised of a zinc finger DNA binding domain and either a domain that activates transcription or a domain that represses transcription. Artificial transcription factors attach to the DNA at a specific binding site as dictated by the construction of the zinc finger DNA binding domain. Once an artificial transcription factor binds to the DNA, if the zinc finger DNA binding domain is physically attached to an activating domain, then transcription proteins become activated and a transcription complex is assembled. If an artificial transcription factor binds to the DNA and the zinc finger DNA binding domain is physically attached to a repressor domain, then this form of artificial transcription factor prevents the assembly of a transcription complex, which results in the adjacent DNA not being able to be transcribed. Zinc finger DNA binding domains can be readily designed to attach to specific sequences of the DNA. Artificial transcription factors may be comprised of protein domains exclusively or a combination of one or more protein domains and one or more non-protein elements. Artificial transcription factors may have a ligand binding domain as part of the molecule.

A ‘deoxyribose’ is a deoxypentose (C₅H₁₀O₄) sugar. Deoxyribonucleic acid (DNA) is comprised of three basic elements: a deoxyribose sugar, a phosphate group and nitrogen containing bases. DNA is a macromolecule made up of two chains of repeating deoxyribose sugars linked by phosphodiester bonds between the 3-hydroxyl group of one and the 5-hydroxyl group of the next; the two chains are held antiparallel to each other by weak hydrogen bonds. DNA strands contain a sequence of nucleotides, which include: adenine, cytosine, guanine or thymine. Adenine is always paired with thymine of the opposite strand, and guanine is always paired with cytosine of the opposite strand; one side or strand of a DNA macromolecule is the mirror image of the opposite strand. Nuclear DNA is regarded as the medium for storing the master plan of hereditary information.

Genes are considered segments of the DNA that represent units of inheritance.

A chromosome exists in the nucleus of a cell and consists of a DNA double helix bearing a linear sequence of genes, coiled and recoiled around aggregated proteins, termed histones. The number of chromosomes varies from species to species. Most Human cells carries twenty two pairs of chromosomes plus two sex chromosomes; two ‘x’ chromosomes in women and one ‘x’ and one ‘y’ chromosome in men. Chromosomes carry genetic information in the form of units which are referred to as genes.

Per J. K. Pal, S. S. Ghaskabi, Fundamentals of Molecular Biology, 2009: ‘The central dogma of molecular biology . . . states that the genes present in the genome (DNA) are transcribed into mRNAs, which are then translated into polypeptides or proteins, which are phenotypes.’ ‘Genome, thus, contains the complete set of hereditary information for any organism and is functionally divided into small parts referred to as genes. Each gene is a sequence of nucleotides representing a single protein or RNA. Genome of a living organism may contain as few as 500 genes as in case of Mycoplasma, or as many as 30,000 genes as in case of human beings.’ Some references cite as many as 100,000 genes may exist in the human genome.

The current understanding of the actual biologic structure of a gene is far more elaborate than the historic standard definition of a gene. A gene appears to be comprised of a number of segments loosely strung together along a particular section of DNA. In general there are three segments associated with a gene which include: (1) the Upstream 5′ flanking region, (2) the transcriptional unit (often referred to as the open reading frame) and (3) the Downstream 3′ flanking region. The Upstream 5′ flanking region is comprised of the ‘enhancer region’, the ‘promoter-proximal region’, and ‘promoter region’. The ‘transcriptional unit’ or open reading frame starts at a location designated ‘transcription start site’ (TSS), which is located in a site called the ‘initiator region’ (inR), which may be described in a general form as Py₂CAPy₅. The transcription unit is comprised of the combination of segments of DNA nucleotides to be transcribed into RNA and spacing units known as ‘introns’ that are not transcribed or if transcribed are later removed post transcription, such that they do not appear in the final RNA molecule. In the case of a gene coding for a mRNA molecule, the transcription unit will contain all three elements of the mRNA, which includes: (1) the 5′ noncoding region, (2) the translational region and (3) the 3′ noncoding region. Interspersed between these regions are introns, which will be either not transcribed or if transcribed removed from the precursor form of mRNA prior to the mRNA reaching its final form. The ‘transcriptional unit’ is generally considered to be the ‘gene’. The Downstream 3′ flanking region contains DNA nucleotides that are not transcribed and may contain what has been termed an ‘enhancer region’. An enhancer region in the Downstream 3′ flanking region is thought to act to promote the gene previously transcribed to be transcribed again.

On either side of the DNA sequencing comprising a gene and its flanking regions, may be inactive DNA which act as boundaries which have been termed ‘insulator elements’. The term ‘upstream’ refers to DNA sequencing that occurs prior to the TSS if viewed from the 5′ end to the 3′ end of the DNA; where the term ‘downstream’ refers to DNA sequencing located after the TSS.

An ‘enhancer region’ may or may not be present in the Upstream 5′ flanking region. If present, the enhancer region helps facilitate the reading of the gene by encouraging formation of the transcription mechanism. If the enhancer is present, it may exist 200 kb to 17,000 kb upstream from the transcription starting site.

The ‘transcription complex’ also referred to as the ‘transcription mechanism’, in humans, is reported to be comprised of over forty separate proteins that assemble together to ultimately function in a concerted effort to transcribe the nucleotide sequence of the DNA into RNA. The transcription complex (TC) includes elements such as ‘general transcription factor Sp1’, ‘general transcription factor NF1’, ‘general transcription factor TATA-binding protein’, ‘TF_{II}D’, ‘basal transcription complex’, and a ‘RNA polymerase protein’ to name only a few of the forty elements that exist. The elements of the transcription mechanism function as (1) a means to recognize the location of the start of a gene, (2) as proteins to bind the transcription mechanism to the DNA such that transcription may occur or (3) as means of transcribing the DNA nucleotide coding to produce a precursor RNA molecule. There are at least three RNA polymerase proteins which include: RNA polymerase I, RNA polymerase II, and RNA polymerase III. RNA polymerase I tends to be dedicated to transcribing genetic information that will result in the formation of rRNA molecules. RNA polymerase II tends to be dedicated to transcribing genetic information that will result in the formation of mRNA molecules. RNA polymerase III appears to be dedicated to transcribing genetic information that results in the formation of tRNAs, small cellular RNAs and viral RNAs.

The ‘promoter proximal region’ is located upstream from the TSS and upstream from the core promoter region. The ‘promoter proximal region’ includes two sub-regions termed the GC box and the CAAT box. The ‘GC box’ appears to be a segment rich in guanine-cytosine nucleotide sequences. The GC box binds to the ‘general transcription factor Sp1’ of the transcription mechanism. The ‘CAAT box’ is a segment which contains the nucleotide sequence ‘GGCCAATCT’ located 75 bps upstream from the transcription start site (TSS). The CAAT box binds to the ‘general transcription factor NF1’ of the transcription mechanism.

The ‘core promoter’ region is considered the shortest sequence at which RNA polymerase II can initiate transcription of a gene The core promoter may include the initiator region (inR) and either a TATA box or a DPE. The inR is the region designated Py₂CAPy₅ that surrounds the transcription start site (TSS). The TATA box is located 25 bps upstream from the TSS. The TATA box acts as a site of attachment of the TF_{II}D, which is a promoter for binding of the RNA polymerase II molecule. The DPE is the ‘downstream promoter element’ that may appear 28 bps to 32 bps downstream from the TSS. The DPE acts as an alternative site of attachment for the TF_{II}D when the TATA box is not present.

The transcription mechanism, also referred to as the transcription complex appears to be comprised of different elements depending upon whether rRNA is being transcribed versus mRNA or tRNA or small cellular RNA or viral RNA. The proteins that assemble to assist RNA Polymerase I with transcribing the DNA to produce rRNA appear different than the proteins that assemble to assist RNA polymerase II with transcribing the DNA to produce mRNA or the proteins that assemble to assist RNA polymerase III with transcribing the DNA to produce tRNA, small cellular RNA or viral RNA. A common protein that appears to be present at the initial biding of all three types of RNA polymerase molecules is TATA-binding protein (TBP). TBP appears to be required to attach to the DNA, which then facilitates RNA polymerase to bind to the promoter along the DNA. TBP assembles with TBP-associated factors (TAFs). Together TBF and 11 TAFs comprise the complex referred to as TF_{II}D, which has been previously mentioned in the above text.

Upstream from the TATA box is the ‘initiator element’, which may be considered as part of the ‘core promoter’ region. The initiator element is a segment of the nuclear DNA that binds the basal transcription complex. The basal transcription complex is comprised of a number of proteins that make initial contact with the DNA prior to the RNA polymerase binding to the transcription mechanism. The basal transcription complex is associated with an activator. The activator is a nuclear signaling protein.

Once the transcription complex is assembled, the transcription complex transcribes the DNA. Transcription of the DNA produces precursor messenger RNA (mRNA), precursor ribosomal RNA (rRNA) or precursor transport RNA (tRNA). In the case of precursor mRNA, the mRNA is further modified, then traverses to the cytoplasm. In the cytoplasm of the cell ribosomes attach to the mRNA. Ribosomes decode mRNA utilizing the process of translation to produce proteins.

A protein is comprised of a string of amino acids. Proteins can be comprised of only a few amino acids, or a large number of amino acids. Large protein molecules are often referred to as a macromolecule. Proteins can be combined together to form molecules comprised of two or more similar amino acid strands or a protein can be can be comprised of two or more different amino acid strands. When more than one protein are combined into a molecule, this may also be referred as a macromolecule. Proteins can be combined with other molecules such as carbohydrates and lipids. When proteins combine with other molecules this is also often referred to as a macromolecule. Cell surface receptors are considered macromolecules and are often comprised of a protein molecule combined with a carbohydrate molecule to produce a glycoprotein.

Diabetes mellitus represents an important health issue that affects a significant portion of the world population. In the United States, about 16 million people suffer from diabetes mellitus. Every year, about 650,000 additional people are diagnosed with this disease. Diabetes mellitus is the seventh leading cause of all deaths.

Diabetes mellitus represents a state of hyperglycemia, a serum blood sugar that is higher than what is considered the normal range for humans. Glucose, a six-carbon molecule, is a form of sugar. Glucose is absorbed by the cells of the body and converted to energy by the processes of glycolysis, the Krebs cycle and phosporylation. Insulin, a protein, facilitates the transfer of glucose from the blood into cells. Normal range for blood glucose in humans is generally defined as a fasting blood plasma glucose level of between 70 to 110 mg/dl. For descriptive purposes, the term ‘plasma’ refers to the fluid portion of blood.

Diabetes mellitus is classified as Type One and Type Two. Type One diabetes mellitus is insulin dependent, which refers to the condition where there is a lack of sufficient insulin circulating in the blood stream and insulin must be provided to the body in order to properly regulate the blood glucose level. When insulin is required to regulate the blood glucose level in the body, this condition is often referred to as insulin dependent diabetes mellitus (IDDM). Type Two diabetes mellitus is noninsulin dependent, often referred to as noninsulin dependent diabetes mellitus (NIDDM), meaning the blood glucose level can be managed without insulin, and instead by means of diet, exercise or intervention with oral medications. Type Two diabetes mellitus is considered a progressive disease, the underlying pathogenic mechanisms including pancreatic Beta cell (also often designated as β-Cell) dysfunction and insulin resistance.

The pancreas serves as an endocrine gland and an exocrine gland. Functioning as an endocrine gland the pancreas produces and secretes hormones including insulin and glucagon. Insulin acts to reduce levels of glucose circulating in the blood. Beta cells secrete insulin into the blood when a higher than normal level of glucose is detected in the serum. For purposes of this description the terms ‘blood’, ‘blood stream’ and ‘serum’ refer to the same substance. Glucagon acts to stimulate an increase in glucose circulating in the blood. Beta cells in the pancreas secrete glucagon when a low level of glucose is detected in the serum.

Glucose enters the body as food and as a result of digestion, glucose enters the blood stream. The Beta cells of the Islets of Langerhans continuously sense the level of glucose in the blood and respond to elevated levels of blood glucose by secreting insulin into the blood. Beta cells produce the protein ‘insulin’ in their endoplasmic reticulum and store the insulin in vacuoles until it is needed. When Beta cells detect an increase in the glucose level in the blood, Beta cells release insulin into the blood from the described storage vacuoles.

Insulin is a protein. An insulin protein consists of two chains of amino acids, an alpha chain and a beta chain, linked by two disulfide (S—S) bridges. One chain, the alpha chain consists of 21 amino acids. The second chain the beta chain consists of 30 amino acids.

Insulin interacts with the cells of the body by means of a cell-surface receptor termed the ‘insulin receptor’ located on the exterior of a cell's ‘outer membrane’, otherwise known as the ‘plasma membrane’. Insulin interacts with muscle and liver cells by means of the insulin receptor to rapidly remove excess blood sugar when the glucose level in the blood is higher than the upper limit of the normal physiologic range. Recognized functions of insulin include stimulating cells to take up glucose from the blood and convert it to glycogen to facilitate the cells in the body to utilize glucose to generate biochemically usable energy, and to stimulate fat cells to take up glucose and synthesize fat.

Diabetes Mellitus may be the result of one or more factors. Causes of diabetes mellitus may include: (1) mutation of the insulin gene itself causing miscoding, which results in the production of ineffective insulin molecules; (2) mutations to genes that code for the ‘transcription factors’ needed for transcription of the insulin gene in the deoxyribonucleic acid (DNA) to create messenger ribonucleic acid (mRNA) molecules, which facilitate the manufacture of the insulin molecule; (3) mutations of the gene encoding for the insulin receptor, which produces inactive or an insufficient number of insulin receptors; (4) mutation to the gene encoding for glucokinase, the enzyme that phosphorylates glucose in the first step of glycolysis; (5) mutations to the genes encoding portions of the potassium channels in the plasma membrane of the Beta cells, preventing proper closure of the channel, thus blocking insulin release; (6) mutations to mitochondrial genes that as a result, decreases the energy available to be used facilitate the release of insulin, therefore reducing insulin secretion; (7) failure of glucose transporters to properly permit the facilitated diffusion of glucose from plasma into the cells of the body.

The insulin molecule is a protein produced by Beta cells located in the pancreas. A ‘pro-insulin messenger RNA’ is created in a Beta cell by a transcription complex transcribing the insulin gene from nuclear DNA. The pro-insulin messenger RNA (mRNA) is modified, then travels out of the cytoplasm. Ribosomes, decode the mRNA to produce insulin. Once the biologically active insulin protein is generated it is stored in a vacuole in the Beta cell to await being released into the blood stream.

Insulin receptors, which appear on the surface of cells, offer binding sites for insulin circulating in the blood. When insulin binds to an insulin receptor, the biologic response inside the cell causes glucose to enter the cell and undergo processing in the cytoplasm. Processed glucose molecules then enter the mitochondria. The mitochondria further process the modified glucose molecules to produce usable energy in the form of adenosine triphosphate molecules (ATP). Thirty-eight ATP molecules may be generated from one molecule of glucose during the process of aerobic respiration. ATP molecules are utilized as an energy source by biologic processes throughout the cell.

The current medical therapeutic approach to the management of diabetes mellitus has produced limited results. Patients with diabetes generally struggle with an inadequate production of insulin, or an ineffective release of biologically active insulin molecules, or a release of an insufficient number of biologically active insulin molecules, or an insufficient production of cell-surface receptors, or a production of ineffective cell-surface receptors, or a production of ineffective insulin molecules that are unable to interact properly with insulin receptors to produce the required biologic effect. Type One diabetes requires administration of exogenous insulin. The traditional approach to Type Two diabetes has generally first been to adjust the diet to limit the caloric intake the individual consumes. Exercise is used as an initial approach to both Type One and Type Two diabetes as a means of up-regulating the utilization of fats and sugar so as to reduce the amount of circulating plasma glucose. When diet and exercise are inadequate in properly managing Type Two diabetes, oral medications are often introduced. The action of sulfonylureas, a commonly prescribed class of oral medication, is to stimulate the Beta cells to produce additional insulin receptors and enhance the insulin receptors' response to insulin. Biguanides, another form of oral treatment, inhibit gluconeogenesis, the production of glucose in the liver, thereby attempting to reduce plasma glucose levels. Thiazolidinediones (TZDs) lower blood sugar levels by activating peroxisome proliferator-activated receptor gamma (PPAR-γ), a transcription factor, which when activated regulates the activity of various target genes, particularly ones involved in glucose and lipid metabolism. If diet, exercise and oral medications do not produce a satisfactory control of the level of blood glucose in a diabetic patient, exogenous insulin is injected into the body in an effort to normalize the amount of glucose present in the serum. Insulin, a protein, has not successfully been made available as an oral medication to date due to the fact that proteins in general become degraded when they encounter the acid environment present in the stomach.

Despite strict monitoring of blood glucose and potentially multiple doses of insulin injected throughout the day, many patients with diabetes mellitus still experience devastating adverse effects from elevated blood glucose levels. Microvascular damage and elevated tissue sugar levels contribute to such complications as renal failure, retinopathy involving the eyes, neuropathy, and accelerated heart disease despite aggressive efforts to maintain the blood sugar within the physiologic normal range using exogenous insulin by itself or a combination of exogenous insulin and one or more oral medications. Diabetes remains the number one cause of renal failure in the United States. Especially in diabetic patients whom are dependent upon administering exogenous insulin into their body, though dosing of the insulin may be four or more times a day and even though this may produce adequate control of the blood glucose level to prevent the clinical symptoms of hyperglycemia; this does not unerringly supplement the body's natural capacity to monitor the blood sugar level minute to minute, twenty-four hours a day, and deliver an immediate response to a rise in blood glucose by the release of insulin from Beta cells as required. The deleterious effects of diabetes may still evolve despite strict and persistent control of the glucose level in the blood stream.

Current treatment approach to managing diabetes may be augmented by the unique approach to utilizing modified viruses as vehicles to transport of nuclear signaling protein molecules into cells in order to increase the production of biologically active insulin. By utilizing modified viruses to transport nuclear signaling protein molecules to facilitate the production of mRNAs, which would then facilitate the assembly of the necessary proteins. A diabetic would require the necessary proteins to adequately control the blood glucose level by utilizing inherent regulatory mechanisms rather than exogenous therapies.

Present medical care is attempting to utilize viruses to deliver genetic information into cells. Research in the field of gene therapy has involved certain naturally occurring viruses. Some of the common viral vectors that have been investigated include: Adeno-associated virus, Adenovirus, Alphavirus, Epstein-Barr virus, Gammaretrovirus, Herpes simplex virus, Letivirus, Poliovirus, Rhabdovirus, Vaccinia virus. Naturally occurring virus vectors are limited to the naturally occurring external probes that are affixed to the outer wall of the virus. The external probes fixed to the outside wall of a virus virion dictate which type of cell the virus can engage and infect. Therefore, as an example, the function of the adenovirus, a respiratory virus, is strictly limited to engaging and infecting specific lung cells. Used as a medical treatment device, the adenovirus can only deliver gene therapy to specific lung cells, which severely limits this vector's usefulness as a deliver device. The therapeutic function of all naturally occurring viral vectors is limited to delivering a DNA or RNA payload to the cell type the viral vector naturally targets as its host cell.

Naturally occurring viruses also have the disadvantage of being susceptible to detection and elimination by a body's immune system. Viruses have been infecting humans for hundreds of thousands of years. A human's innate immune system is very efficient at detecting the presence of most naturally occurring viruses when such a virus is inside the body. The human immune system is quite capable of generating a vigorous response to most intruding viruses, attacking and neutralizing virus virions whenever a virus virion physically exists are outside the exterior wall of the virus's host cell. If gene therapy in its current state were to become a clinical therapeutic tool, the naturally occurring viruses selected for gene therapy research will have limited effectiveness due the fact that once the viral vector is introduced into the body, the body's the immune system will quickly engage and eliminate the viral vectors, possibly before the vector is able to deliver its payload to its host cell or target cell.

Cichutek, K., 2001 (U.S. Pat. No. 6,323,031 B1) teaches preparation and use of novel lentiviral SiVagm-derived vectors for gene transfer into selected cell types, specifically into proliferatively active and resting human cells.

Cichutek teaches that it is indeed plausible to re-configure an existing virus and use it as a transport vehicle, though Cichutek's specification and claims are too limited to describe a method that will work for all cell types, if indeed if it will work for any cell type.

Cichutek describes vectors for ‘gene transfer’; in the claims the language that is used is ‘genetic information’. Cichutek's Claim 1 of the cited patent states ‘A propagation-incompetent SIVagm vector comprising a viral core and a viral envelope, wherein the viral core comprises a simian immunodeficiency virus (SIVagm) viral core of the African vervet monkey Chlorocebus.’ Cichutek's does not describe in his claims any further details of the intended payload other than the stating ‘SIVagm viral core’ in claim 1; in claims 5 & 6 Cichutek describes only ‘genetic information’. Transfer of ‘genetic information’ dramatically limits the useful application of Cichutek's patent in the treatment of medical diseases.

Cichutek does not claim the use of specific glycogen probes to target specific types of cells. Cichutek's approach is dependent upon the probes naturally present on the viral vectors reported in the patent, which will direct the viral vectors to only those cells the viruses naturally use as their host cell. Cichutek's approach is very restrictive, limited to gene transfer to only cells the viruses use as their natural host cell.

It is questionable that Cichutek's approach as described in the specification and claims is feasible. Cichutek's claim 4, states ‘The SIVagm vector of claim 1, wherein the viral envelope further comprises a single chain antibody (scFv) or a ligand of a cell surface molecule.’ By use of the words ‘a’ and ‘or’ in the claim, the claim is limited in the singular, meaning Cichutek claims a single chain antibody or a singular ligand. Singular type antibodies or ligands can be used for cell to cell communication, but to open an access portal into a cell and insert a payload into the cell requires two different types antibodies or ligands. As an example human immunodeficiency virus requires the use of both the gp120 and gp41 probes to open a portal into a T-Helper cell and insert its genome into the T-Helper cell. The gp120 probe engages the CD4+ cell-surface receptor on the T-cell. Once the gp120 probe has successfully engaged a CD4+cell-surface receptor on the target T-Helper cell, then the HIV virion's gp41 probe can engage either a CXCR4 or a CCR5 cell-surface receptor on the T-Helper cell in order to open up an access portal for HIV to insert its genome into a T-Helper cell. It is well documented in the medical literature that a genetic defect leading to an abnormality in the CXCR4 cell-surface receptor prevents HIV virions from opening an access portal and inserting its genetic payload into such T-Helper cells. This genetic defect offers the subset of people carrying the genetic defect resistance to HIV infection. This example demonstrates the need for at least two types of glycoprotein probes to be present on the surface of a viral vector in order for a viral vector to be capable of opening an access portal and delivering the payload the vector carries into its host cell or target cell.

A delivery system that offered a defined means of targeting specific types of cells, would invoke minimal or no response by the innate immune system when present in the body, and a delivery system that would be capable of inserting into cells a wide variety of nuclear signaling proteins would significantly improve the current medical treatment options available to clinicians treating patients.

The solution to arriving at a versatile, workable delivery system that will meet the needs of a number of medical treatments involves three important elements. These elements include:
(1) configurable glycoprotein probes whereby more than one type of glycoprotein probe is to be used to engage and access specific target cell types in order to successfully deliver a payload into a specific cell type,
(2) an exterior envelope comprised of a protein shell or lipid layer expressing the least number of cell-surface markers, such as the use of a stem cell to act as the host cell to manufacture the delivery devices,
(3) configuring the core of the vector to enable it to carry and deliver nuclear signaling proteins.

Viruses are obligate parasites. Viruses simply represent a carrier of genetic material and by themselves viruses are unable to replicate or carry out any form of biologic function outside their host cell. A ‘virion’ refers to the physical structure of a single complete virus as it exists outside of the host cell. Viruses are generally comprised of one or more nested shells constructed of one or more layers of protein, some with a lipid outer envelope, a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's probes is to locate and engage a host cell's receptors. The virus's surface probes are designed to detect, make contact with and functionally engage one or more receptors located on the exterior of a cell type that will offer the virus the proper environment in which to construct copies of itself. A host cell provides the virus the proper biologic machinery for the virus to successfully replicate itself. Once the virus's genome is inside the host cell, the viral genome takes command of the cell's production machinery and causes the host cell to generate copies of the virus. As the viral copies exit the host cell, these virions set off in search of other host cells to infect.

Naturally occurring viruses exist in a number of differing shapes. The shape of a virus may be rod or filament like, icosahedral, or complex structures combining filament and polygonal shapes. Viruses generally have their outer wall comprised of a protein coat or an envelope comprised of lipids.

An outer envelope comprised of lipids may be in the form of one or two phospholipid layers. When the outer envelope is comprised of two phospholipid layers this is termed a lipid bilayer. A phospholipid is a composite molecule comprised of a polar or hydrophilic region on one end and a nonpolar or hydrophobic region on the opposite end. A lipid bilayer covering a virus, like the membrane of a cell, is constructed with the hydrophilic region of one of the phospholipid layers pointed toward the exterior of the virion and the hydrophilic region of the second phospholipid layer pointed inward toward the center of the virus virion; with the hydrophobic regions of each of the two lipid layers pointed toward each other. The outer envelope of some forms of virus may be comprised of an outer lipid layer or lipid bilayer affixed to a protein matrix for support, the protein matrix being located closer to the center of the virus virion than the lipid layer or lipid bilayer.

Spherical viruses are generally spherical in shape and may be comprised of an outer envelope and one inner shell or an outer envelope and multiple inner shells. Inner shells are approximately spherical in shape; this is because the proteins comprising the protein matrix shell have an irregular shape to their structure. In the case of a spherical virus with an outer envelope and one inner shell, the inner shell is often referred to as a nucleocapsid shell comprised of numerous capsid proteins attached to each other. In the case of a spherical virus being comprised of an outer envelope and multiple inner shells, the outermost inner viral shells may be referred to as comprised of a quantity of matrix proteins, where the innermost shell is referred to as a nucleocapsid and is comprised of a quantity of capsid proteins. The inner protein shells are nested inside each other.

Viruses carry genetic material in the form of deoxyribonucleic acid (DNA) or ribonucleic acid (RNA) in their nucleocapsid often referred to as the core. A virus is therefore generally considered to be a DNA virus if its genome is comprised of DNA or the virus is considered a RNA virus if its genome is comprised of RNA. Viruses may also carry enzymes as part of their payload. An enzyme such as ‘reverse transcriptase’ transforms a RNA viral genome into DNA. Protease enzymes modify the viral genome once it has entered a host cell. An integrase enzyme assists a DNA viral genome with insertion into the host cell's nuclear DNA. The payload is carried inside the virus's nucleocapsid shell.

The probes attached to the exterior of a virus are constructed to engage specific cell-surface receptors on specific cell types in the body. Only a cell that expresses cell-surface receptors that are capable of being engaged by the probes of a specific virus can act as a host for the virus. Viruses often use two probes to access a host cell. The first probe makes an initial attachment to the host cell, while the action of the virus's second probe often in conjunction with the action of the first probe cause an access portal to be created in the host cell's exterior plasma membrane. Once an access portal is formed, the virus inserts the contents of its payload into the host cell. Once the virus's genome is inside the cytoplasm of the host cell, any enzymes that accompanied the viral genome into the cell, may begin to modify or assist the virus's genome with infecting and taking control of the host cell's biologic functions.

Probes are attached to the exterior envelope of a virus virion. Probes may be in the form of a protein structure or may be in the form of a glycoprotein molecule. For viruses constructed with a protein matrix as its outer envelope, the probes tend to be protein structures. A portion of the protein structure probe is fixed or anchored in the protein matrix, while a portion of the protein structure probe extends out and away from the protein matrix. The portion of the protein structure probe extending out away from the virus virion is referred to as the ‘exterior domain’, the portion anchored in the protein matrix is the ‘transcending domain’. Some protein probes have a third segment that extends through the envelope and exists inside the virus virion, which is referred to as the ‘interior domain’. The exterior domain of a protein structure probe is intended to engage a specific cell-surface receptor on a biologically active cell the virus is targeting as its host cell.

Viruses that utilize a lipid layer as the outer envelope, are constructed with probes that tend to be glycoproteins. A glycoprotein is comprised of a protein segment and a carbohydrate segment. The carbohydrate segment of the glycoprotein molecule is fixed or anchored in the lipid layer of the outer envelope, while the protein segment extends outward and away from the outer envelope. The protein portion of a glycoprotein probe that extends outward and away from the outer envelope of a virus virion is intended to engage a cell-surface receptor on a biologically active cell the virus is targeting as its host cell.

Some forms of viruses that utilize a lipid layer as its envelope use protein structure probes. In this case, the portion of the protein structure probe that extends outward and away from the outer envelope is the ‘exterior domain’, the portion that is anchored in the lipid layer is the ‘transcending domain’ and again some protein structure probes have an ‘interior domain’ that exist inside the virion, which may also help anchor the protein structure probe to the virion. The exterior domain of a protein structure probe that extends outward and away from the outer envelope of a virus virion is intended to engage a cell-surface receptor on a biologically active cell the virus is targeting as its host cell.

When a virus carries a DNA payload and the viral DNA is inserted into the host cell, the virus's DNA travels to the host cell's nucleus and is known to become inserted into the host cell's own native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate use of the viral RNA by having the RNA converted to DNA. Once the viral RNA has been converted to DNA, the virus's DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the nuclear DNA and simply utilize portions of the viral genome to act as messenger RNA. RNA viruses that bypass the host cell's DNA, cause the cell in general to generate copies of the necessary parts of the virus directly from the virus's RNA genome.

The human immunodeficiency virus (HIV) has an outer envelope comprised of a lipid bilayer. The lipid bilayer covers a protein matrix consisting of p17^{gag} proteins. Inside the p17^{gag} protein is nested a nucleocapsid comprised of p24^{gag} proteins. Inside the nucleocapsid HIV carries its payload. HIV's genetic payload consists of two single strands of RNA. In addition to the two strands of HIV RNA, there are proteins that are carried in the core of the nucleocapsid along with the two RNA strands. These proteins include ‘reverse transcriptase’, ‘Integrase’ and ‘protease’ molecules.

The T-Helper cell acts as HIV's host cell. HIV locates its host by utilizing at least two different types of probes located on its envelope. The HIV virion utilizes two types of glycoprotein probes affixed to the outer surface of its exterior envelope to engage a T-Helper cell. HIV utilizes a glycoprotein probe 120 to locate a CD4 cell-surface receptor on a T-Helper cell. Once an HIV glycoprotein 120 probe has successfully engaged a CD4 cell surface-receptor on a T-Helper cell a conformational change occurs in the probe and a glycoprotein 41 probe is exposed. The glycoprotein 41 probe's intent is to engage a CXCR4 or CCR5 cell-surface receptor on the same T-Helper cell. Once a glycoprotein 41 probe on the HIV virion successfully engages a CXCR4 or CCR5 cell-surface receptor, the HIV virion opens an access portal through the T-Helper cell's outer membrane.

Once the HIV virion has opened an access portal through the T-Helper cell's outer plasma membrane, the HIV virion inserts two positive strand RNA molecules it carries into the T-Helper cell. Each RNA strand is approximately 9500 nucleotides in length. Inserted along with the RNA strands are the enzymes reverse transcriptase, protease and integrase. Once the virus's genome gains access to the interior of the T-Helper cell, in the cytoplasm the pair of RNA molecules are transformed to deoxyribonucleic acid by the reverse transcriptase enzyme. Following modification of the virus's genome to DNA, the virus's genetic information migrates to the host cell's nucleus. In the nucleus, with the assistance of the integrase protein, the HIV's DNA becomes inserted into the T-Helper cell's native DNA. When the timing is appropriate, the now integrated viral DNA is decoded by the host cell's polymerase molecules and the virus's genetic information commands certain cell functions to carry out the replication process to construct copies of the human deficiency virus.

The outer layer of the HIV virion is comprised of a portion of the T-Helper cell's outer cell membrane. In the final stage of the replication process, as a copy of the HIV capsid, carrying the HIV genome, buds through the host cell's cell membrane the capsid acquires as its exterior envelope, a wrapping of lipid bilayer from the host cell's cell membrane. In the case of HIV, since the surface of the pathogen is covered by an envelope comprised of lipid bilayer taken from the host T-Helper cells, this feature allows the HIV virion the capacity to eluded the immune systems, since the cells comprising the immune system may find it difficult to tell the difference between the surface of an infectious HIV virion and the surface characteristics of a noninfected T-Helper cell.

The Hepatitis C virus (HCV) is a positive sense RNA virus, meaning a type of RNA that is capable of bypassing the need for involving the host cell's nucleus by having its RNA genome function as messenger RNA. Hepatitis C infects liver cells. The Hepatitis C viral genome becomes divided once it gains access to the interior of a liver host cell. Portions of the subdivisions of the Hepatitis C genome directly interact with ribosomes to produce proteins necessary to construct copies of the virus.

HCV belongs to the Flaviviridae family and is the only member of the Hepacivirus genus. There are considered to be at least 100 different strains of Hepatitis C virus based on genome sequencing variability.

HCV is comprised of an outer lipoprotein envelope and an internal nucleocapsid. The genetic payload is carried within the nucleocapsid. In its natural state, present on the surface of the outer envelope of the Hepatitis C virus are probes that detect receptors present on the surface of liver cells. The glycoprotein E1 probe and the glycoprotein E2 probe have been identified to be affixed to the surface of HCV. The E2 probe binds with high affinity to the large external loop of a CD81 cell-surface receptor. CD81 is found on the surface of many cell types including liver cells. Once the E2 probe has engaged the CD81 cell-surface receptor, cofactors on the surface of HCV's exterior envelope engage either or both the low density lipoprotein receptor (LDLR) or the scavenger receptor class B type I (SR-BI) present on the liver cell in order to effect the mechanism to facilitate HCV breaching the cell membrane and inserting its RNA genome payload through the plasma cell membrane of the liver cell into the liver cell. Upon successful engagement of the HCV surface probes with a liver cell's cell-surface receptors, HCV inserts the single strand of RNA and other payload elements it carries into the liver cell targeted to be a host cell. The HCV RNA genome then interacts with enzymes and ribosomes inside the liver cell in a translational process to produce the proteins required to construct copies of the protein components of HCV. The HCV genome undergoes a method of transcription to replicate copies of the virus's RNA genome. Inside the host, pieces of the HCV virus are assembled together and ultimately loaded with a copy of the HCV genome. Replicas of the original HCV then escape the host cell and migrate the environment in search of additional host liver cells to infect and continue the replication process.

The HCV's naturally occurring genetic payload consists of a single molecule of linear positive sense, single stranded RNA approximately 9600 nucleotides in length. By means of a translational process a polyprotein of approximately 3000 amino acids is generated. This polyprotein is cleaved post translation by host and viral proteases into individual viral proteins which include: the structural proteins of C, E1, E2, the nonstructural proteins NS1, NS2, NS3, NS4A, NS4B, NS5A, NS5B, p7 and ARFP/F protein. Hepatitis C virus's proteins direct the host liver cell to construction copies of the Hepatitis C virus. A membrane associated replicase complex consisting of the virus's nonstructural proteins NS3 and NS5B facilitate the replication of the viral genome. The membrane of the endoplasmic reticulum appears to be the site of protein maturation and viral assembly. Once copies of the Hepatitis C Virus are generated, they exit the host cell and each copy of HCV migrates in search of another appropriate liver cell that will act as a host to continue the replication process.

Hepatitis C virus life-cycle demonstrates that copies of a virus virion can be generated by inserting RNA into a host cell that functions as messenger RNA in the host cell. The Hepatitis C viral RNA genome functions as messenger RNA, acting as the template in conjunction with the biologic machinery of a host cell to produce the components that comprise copies of the Hepatitis C virion and the Hepatitis C viral RNA provides the biologic instructions to assemble the components into complete copies of the Hepatitis C virions. The Hepatitis C virus life-cycle clearly demonstrates that viral virions can be manufactured by a host cell without involving the nucleus of the cell.

Deciphering the existence, replication and behavior of viruses provides clear examples of several fundamental concepts, which include: (1) Viruses target specific cells in the body by means of identifying and engaging such target cells utilizing the probes projecting outward from the virus's exterior shell to make contact with cell-surface receptors located on the surface of the target cells, and (2) Viruses are capable of carrying various types of payloads including DNA, RNA and a variety of proteins.

Current gene therapy approach to attempting to deliver a payload to cells in the body use modified forms of existing viruses to act as transport devices to deliver genetic information. This approach is severely limited by restricting the virus virion to the target only cells the viral vector naturally seeks out and infects. Current gene therapy approach is further limited by using the pre-existing size of naturally occurring viruses, rather than being able to modify the size of the structure to be able to tailor the volumetric carrying capacity of the payload portion of the modified virus. Further gene therapy is restricted to utilizing naturally occurring viruses to deliver only genetic information; it has not previously been appreciated by those skilled in the art that virus-like transport devices might deliver to a variety of specific cell types a wide variety of differing payloads such as signaling proteins.

A dramatic, not previously recognized by those expert in the art is the need to develop a transport vehicle that can be fashioned to seek out specific types of cells and deliver to these cells nuclear signals. The exterior envelope of a transport should be constructed so as not to alert the immune system of its presence to prevent rejection of the vehicles. Transport vehicles should be capable of being configured to target any specific cell type and engage and deliver their payload only to that specific cell type. To this point, no such device has been conceived.

### BRIEF SUMMARY OF THE INVENTION

Utilization of configurable microscopic medical payload delivery devices to deliver nuclear signaling proteins to specific cell types facilitates a dramatic new approach to medical care. By selecting the type of probes that are present on the surface of the configurable microscopic medical payload delivery devices, specific types of cells can be targeted. By delivering nuclear signaling proteins to specific cell types, genes can be activated or inactivated in those specific cell types. A wide variety of medical conditions are treatable by utilizing this new and unique approach.

### DETAILED DESCRIPTION

The future of medical treatment will be the widespread utilization of configurable microscopic medical payload delivery devices (CMMPDD) to deliver nuclear signaling proteins directly to targeted cell types in the body.

For purposes of this text an ‘external envelope’ refers to the outermost covering of a virus or a virus-like transport device or a configurable microscopic medical payload delivery device. The external envelope may be comprised of a lipid layer, a lipid bilayer, the combination of a lipid layer affixed to a protein matrix or the combination of a lipid bilayer affixed to a protein matrix.

For purposes of this text an ‘internal shell’ refers to a protein matrix shell nested inside the external envelope. The inner most protein matrix shell is termed the nucleocapsid. The proteins that comprise the nucleocapsid are termed capsid proteins. In the center or core of the nucleocapsid is where the payload is carried.

For purposes of this text ‘external probes’ are molecular structures that are utilized to locate and engage cell-surface receptors on biologically active cells. External probes are generally comprised of a portion which is anchored or fixed in the external envelope and a second portion that extends out and away from the external envelope. External probes may be comprised solely of a protein structure or an external probe may be a glycoprotein molecule.

For purposes of this text ‘glycoprotein molecule’ refers to a molecule comprised of a carbohydrate region and a protein region. Glycoprotein molecules that act as probes are generally anchored or fixed to a lipid layer utilizing the carbohydrate portion of the molecule as an anchor. The protein portion of the glycoprotein molecule which extends outward and away from the exterior envelope the glycoprotein has been affixed such that the protein region may function as a probe to locate and attach to the cell-surface receptor it was created to engage.

The concept of configurable microscopic medical payload delivery devices is modeled after naturally existing viruses. Configurable microscopic medical payload delivery devices in general are spherical in shape; though other shapes may be used as function might warrant the use of a particular shape. The spherical configurable microscopic medical payload delivery devices are comprised of an exterior envelope and one or more inner nested protein shells. A quantity of exterior protein structure probes and/or glycoprotein probes are anchored in the exterior lipid envelope and extend out and away from the exterior lipid envelope. Nesting of protein shells refers to progressively smaller diameter shells fitting snugly inside protein shells of a larger diameter. Inside the inner most protein shell, referred to as the nucleocapsid, is a cavity referred to as the core of the device. The core of the device is the space where the medically therapeutic payload the device carries is located.

Configurable microscopic medical payload delivery devices are generated to target certain specific cell types in the body. Configurable microscopic medical payload delivery devices target specific cell types by the configuration of probes affixed to the exterior envelope of the CMMPDD. By affixing specific probes to the exterior envelope of the CMMPDD, these probes intended to engage and attach only to specific cell-surface receptors located on certain cell types in the body, the CMMPDD will deliver its payload only to those cell types that express compatible and engagable specific cell-surface receptors. In a similar fashion where the exterior probes of a naturally occurring virus engage specific cell-surface receptors present on the surface of the virus's host cell and only the designated host cell, the CMMPDD's exterior probes are configured to engage cell-surface receptors on a specific type of target cell. In this manner, the payload of medication or biologic tools carried by CMMPDD will be delivered only to specific types of cells in the body. The exterior probes on the surface of a CMMPDD will vary as needed so as to effect the CMMPDD delivery of payloads to cell types as needed to effect a medical treatment.

The size of configurable microscopic medical payload delivery devices is to depend upon the volume size of the payload the CMMPDD is required to carry and deliver to a target cell. The size of a CMMPDD is dependent upon the diameter of the inner protein matrix shells. The diameter of each inner protein matrix shell is governed by the number of protein molecules utilized to construct the protein matrix shell at the time the protein matrix shell is generated. Increasing the number of proteins that comprise a protein matrix shell, increases the diameter of the protein matrix shell. The external lipid envelope wraps around and covers the outermost protein matrix shell. The larger the volume of the core of the CMMPDD, the greater the physical size payload the CMMPDD is able to carry. The size of the configurable microscopic medical payload delivery device is to be the size of cell (approximately 10⁻⁴ m in diameter) or less, generally detectable by a light microscope or, as needed, an electron microscope. The size of the CMMPDD is not to be too large such that it would generate a burden to the body by damaging organ tissues through clogging blood vessels, and the maintaining a small enough size that the CMMPDD can be properly disposed of by the body once the CMMPDD has delivered its payload to its target cell. The dimensions of each type of CMMPDD are to be tailored to the mission of the CMMPDD, which takes into account the type of target cell, the size of the payload that is to be delivered to the target cells and the length of time the CMMPDD may engage the target cell.

Being enveloped in an external lipid layer, configurable microscopic medical payload delivery devices possess the advantage of having their exterior appear similar to the plasma membrane that acts as an outside covering for the cells that comprise the body. By appearing similar to existing plasma membranes, the CMMPDDs appear similar to naturally occurring structures found in the body, affording the CMMPDD the capability to avoid detection by a body's immune system because the exterior of the CMMPDD mimics the cells comprising the body and the surveillance cells of the immune system find it difficult to discern between the CMMPDD and naturally occurring cells comprising the body.

To carry out the process of manufacturing a configurable microscopic medical payload delivery device, a primitive cell such as a stem cell is selected. The reason for utilizing primitive cells such as stems cells as the host cell, is that the CMMPDD acquires its outer envelope from the host cell and the more primitive the host cell, the fewer in number the identifying protein markers are present on the surface of the CMMPDD. The fewer the identifying surface proteins present on the outer envelope of the CMMPDD, the less likely a body's immune system will identify the CMMPDD as an invader and therefore less likely the body's immune system will react to the presence of the CMMPDD and reject the CMMPDD by attacking and neutralizing the CMMPDD.

Stem cells used as host cells to manufacture quantities of CMMPDD product are selected per histocompatibility markers present on their surface. Certain histocompatibility markers present on the surface of the final CMMPDD product will be less likely to cause a reaction in a specific patient based on the genetic profile of the patient's histocompatibility markers. A similar histocompatibility match is done when donor organs are selected to be given to recipients to avoid rejection of the donor organ by the recipient's immune system.

The selected stem cell used to manufacture configurable microscopic medical payload delivery devices goes through several steps of maturation before it is capable of generating therapeutic CMMPDD product. Messenger RNA would be inserted into the host stem cell that would code for the general physical outer structures of the CMMPDD. Messenger RNA would be inserted into the host that would generate surface probes that would target the surface receptors on specifically target cells. Messenger RNA would be inserted into the host that would be used to generate the payload of nuclear signaling proteins. Similar to how copies of a naturally occurring virus, such as the Hepatitis C virus or HIV, are produced, assembled and released from a host cell, copies of the CMMPDD would be produced, assembled and released from a host cell. Once released from the host cell, the copies of the CMMPDD would be collected, then pooled together to produce a therapeutic dose that would result in a medically beneficial effect.

The construction of the configurable microscopic medical payload delivery devices is performed by taking stem cells and inserting modified viral genetic programming into the stem cells. Stem cells are chosen as the host cell due to the low number of surface markers, which leads to less antigenicity in configurable microscopic medical payload delivery devices when the configurable microscopic medical payload delivery devices are released by the host cells and wrapped in an outer envelope comprised of the host cells' plasma membrane.

The stem cells used as host cells are suspended in a broth of nutrients and are kept at an optimum temperature to govern the rate of production of the CMMPDD product. Similar to the natural production of the Hepatitis C virus, the configurable microscopic medical payload delivery devices ‘production genome’ is introduced into the host stem cells. The configurable microscopic medical payload delivery devices production genome carries genetic instructions to cause the host cells to manufacture the configurable microscopic medical payload delivery devices' outer protein wall, the inner protein matrixes, the surface probes the configurable microscopic medical payload delivery device is to have affixed to its outer envelope and the nuclear signaling proteins the configurable microscopic medical payload delivery devices are to carry; and the instructions to assemble the various pieces into the final form of the configurable microscopic medical payload delivery devices and the instructions to activate the budding process. The resultant configurable microscopic medical payload delivery devices are collected from the nutrient broth surrounding the host cells and placed together into doses to be used as a treatment for a medical disease.

The ‘production genome’ are an array of messenger RNAs that are directly translated by the host cell's internal enzymes. The production genome dictates the characteristics of the final version of the CMMPDD that buds from the host stem cell and is released and is to be utilized as a medical treatment. The production genome is specifically tailored to code for the surface probes that will seek and engage a specific type of target cell. The production genome also carries the instructions to code for the production of the type of nuclear signaling proteins to be delivered to the specific type of target cell. The ‘production genome’ varies depending upon the configuration of the CMMPDD and the type of nuclear signaling proteins the CMMPDD will transport to effect a specific medical treatment on a specific type of cell.

The configurable microscopic medical payload delivery device transporting nuclear signaling proteins represents a very versatile medical treatment delivery device. There are an estimated 30,000 to 100,000 genes located in the human genome. CMMPDD could be used to deliver nuclear signaling proteins to activate or inactivate any of the chromosomal genes in any specific cell type in the body.

As an example of this method, to treat diabetes mellitus utilizing configurable microscopic medical payload delivery devices to deliver to Beta cells messenger RNA coded to produce insulin, the following production process is followed in the lab: (1) human stem cells are selected. (2) Into the selected stem cells is placed the production genome constructed, in this case, specifically as a means to treat diabetes mellitus. The RNA production genome contains genetic instructions to cause the host stem cells to manufacture the configurable microscopic medical payload delivery devices' outer protein wall, the inner protein matrix, surface probes to include glycoprotein probes that engage the GPR40 cell-surface receptor present on the surface of Beta cells located in the Islets of Langerhans in the pancreas, and the payload of nuclear signaling proteins, in this case the nuclear signaling proteins to activate the production of the insulin molecules in Beta cells; and the biologic instructions to assemble the components into the final form of the configurable microscopic medical payload delivery devices; and the biologic instructions to activate the budding process. (3) Upon insertion of the RNA production genome dedicated to producing a nuclear signaling proteins configured to activate the genes to generate messenger RNA that will result in the production of insulin, into the host stem cells, host stem cells respond by (i) simultaneously translating the different segments of the RNA production genome to produce the proteins that comprise the exterior protein wall, the inner protein matrix molecules, the surface probes to seek out and engage Beta cells, the nuclear signaling protein payload to produce insulin, and (ii) decoding the RNA instructions to assemble the components into the configurable microscopic medical payload delivery devices. (4) Upon assembly, the configurable microscopic medical payload delivery devices bud through the cell membrane of the host stem cell. (5) At the time of the budding process, the configurable microscopic medical payload delivery devices acquire an outside envelope wrapped over the outer protein shell, this outer envelope comprised of a portion of the plasma membrane from the host stem cell as the configurable microscopic medical payload delivery devices exit the host cell. (6) The resultant configurable microscopic medical payload delivery devices are collected from the nutrient broth surrounding the host stem cells. (7) The configurable microscopic medical payload delivery devices are washed in sterile solvent to remove contaminants. (8) The configurable microscopic medical payload delivery devices are removed from the sterile solvent and suspended in a hypoallergenic liquid medium. (9) The configurable microscopic medical payload delivery devices are separated into individual quantities to facilitate storage and delivery to physicians and patients. (10) The configurable microscopic medical payload delivery devices transported in the hypoallergenic liquid medium is administered to a diabetic patient per injection in a dose that is tailored to receiving patient's requirement to produce sufficient amount of insulin to control the blood sugar. (11) Upon being injected into the body, the configurable microscopic medical payload delivery devices migrate to the Beta cells located in the Islets of Langerhans by means of the patient's blood stream. (12) Upon the configurable microscopic medical payload delivery devices reaching the Beta cells, the configurable microscopic medical payload delivery devices engage the cell-surface receptors located on the Beta cells and insert the payload they carry into the Beta cells. The payload, in this case being nuclear signaling proteins to activated the genes responsible for the Beta cell's production of insulin molecules. The increase in insulin production by Beta cells successfully manages diabetes mellitus.

### CONCLUSIONS, RAMIFICATION, AND SCOPE

Accordingly, the reader will see that the configurable microscopic medical payload delivery device to deliver nuclear signaling proteins to specific targeted cell types provides advantages over existing art by (1) being a delivery device that seeks out specific types of cells, (2) by being a delivery device that is versatile enough to deliver a variety of potential nuclear signaling proteins to accomplish various medical treatments and (3) by being a delivery device constructed with a surface envelope that will avoid detection by the innate immune system so as not to activate the immune system to its presence; for these reasons this represents a new and unique medical delivery device that has never before been recognized nor appreciated by those skilled in the art.

Although the description above contains specificities, these should not be construed as limiting the scope of the invention but as merely providing illustrations of some of the presently preferred embodiments of the invention.

Thus the scope of the invention should be determined by the appended claims and their legal equivalents, rather than by the examples given.

What is claimed:

1. A configurable microscopic medical payload delivery device comprised of:
(a) an exterior envelope,
(b) a quantity of interior shells,
(c) a quantity of configurable exterior probes attached in a manner a segment of said exterior probes is to project out and away from said exterior envelope, while a segment of said exterior probes is embedded in said exterior envelope, and
(d) a quantity of nuclear signaling proteins,
whereby said configurable microscopic medical payload delivery device is intended to deliver said quantity of nuclear signaling proteins to a specific cell type in order to produce a medically beneficial effect,
whereby said exterior probes are intended to engage specific cell-surface receptors on said specific cell type,
whereby said interior shells are versatile enough in their construction to carry within the nucleocapsid a wide variety of nuclear signaling proteins to said specific cell types.

2. The configurable microscopic medical payload delivery device in claim 1 wherein said external envelope is selected from the group consisting of a lipid layer, a lipid bilayer, a protein matrix, a lipid layer affixed to a protein matrix, and a lipid bilayer affixed to a protein matrix.

3. The configurable microscopic medical payload delivery device in claim 1 wherein said quantity of internal shells are comprised of a quantity of nested sphere-like concentric protein matrix structures.

4. The configurable microscopic medical payload delivery device in claim 1 wherein said exterior probes are comprised of a quantity of protein molecules.

5. The protein molecules in claim 4 wherein said protein molecules are comprised of a segment of said protein molecules which extends outward and away from said exterior envelope, attached to a segment of said protein molecule which is embedded in said exterior envelope to hold said protein molecule affixed to said exterior envelope,
whereby said segment of said protein molecules which extends outward and away from said exterior envelope is intended to engage said specific cell-surface receptors on said specific cell type.

6. The protein molecules in claim 4 wherein said protein molecules are comprised of a plurality of protein molecules,
whereby, at least two differing configurations of said protein molecules may be needed to successfully engage said specific cell type with one type of configuration of said protein molecule engaging one type of said specific cell-surface receptor, while a differing type of configuration of said protein molecule is required to engage a differing type of said specific cell-surface receptor in order for said configurable microscopic medical payload delivery device to insert said quantity of nuclear signaling proteins said configurable microscopic medical payload delivery device carries into intended said specific cell type.

7. The configurable microscopic medical payload delivery device in claim 1 wherein said exterior probes are comprised of a quantity of glycoprotein molecules.

8. The glycoprotein molecules in claim 7 wherein said glycoprotein molecules are comprised of a protein segment which extends outward and away from said exterior envelope, which is attached to a carbohydrate segment, said carbohydrate segment being embedded in said exterior envelope to hold said glycoprotein molecule affixed to said exterior envelope,
whereby said protein segment which extends outward and away from said exterior envelope is intended to engage said specific cell-surface receptor on said specific cell type.

9. The glycoprotein molecules in claim 7 wherein said glycoprotein molecules are comprised of a plurality of glycoprotein molecules,
whereby, at least two differing configurations of said glycoprotein molecules may be needed to successfully engage said specific cell type with one type of configuration of said glycoprotein molecule engaging one type of said specific cell-surface receptor, while a differing type of configuration of said glycoprotein molecule is required to engage a differing type of said specific cell-surface receptor in order for said configurable microscopic medical payload delivery device to insert said quantity of nuclear signaling proteins into said specific cell type.

10. The nuclear signaling proteins in claim 1 wherein said nuclear signaling proteins are selected from the group consisting of nuclear receptors, nuclear binding proteins, and artificial transcription factors.

11. A configurable medical treatment payload delivery means for inserting medically beneficial payloads into predetermined biologically active cells of a particular type comprising:
(a) an exterior envelope,
(b) a quantity of interior shells,
(c) a quantity of exterior probes attached to said exterior envelope, and
(d) a quantity of nuclear signaling proteins,
whereby said configurable medical treatment payload delivery means is intended to deliver medically therapeutic payloads to said predetermined biologically active cells of a particular type in order to produce a medically beneficial effect,
whereby said exterior probes are intended to engage specific cell-surface receptors on said predetermined biologically active cells of a particular type,
whereby said interior shells are versatile enough in their construction to carry within the nucleocapsid a wide variety of said nuclear signaling proteins to said predetermined biologically active cells of a particular type.

12. The configurable medical treatment payload delivery means in claim 11 wherein said external envelope is selected from the group consisting of a lipid layer, a lipid bilayer, a protein matrix, a lipid layer affixed to a protein matrix, and a lipid bilayer affixed to a protein matrix.

13. The configurable medical treatment payload delivery means in claim 11 wherein said internal shell is comprised of a quantity of nested concentric protein matrix sphere-like structures.

14. The configurable medical treatment payload delivery means in claim 11 wherein said exterior probes are comprised of a quantity of protein molecules.

15. The protein molecules in claim 14 wherein said protein molecules are comprised of a segment of said protein molecule which extends outward and away from said exterior envelope, which is attached to a segment of said protein molecule embedded in said exterior envelope to hold said protein molecule affixed to said exterior envelope,
whereby said segment of said protein molecule which extends outward and away from said exterior envelope is intended to engage said specific cell-surface receptor on said predetermined biologically active cells of a particular type.

16. The protein molecules in claim 14 wherein said protein molecules are comprised of a plurality of protein molecules,
whereby, at least two differing configurations of said protein molecules may be needed to successfully engage said predetermined biologically active cells of a particular type with one type of configuration of said protein molecule engaging a type of said specific cell-surface receptor, while a differing type of configuration of said protein molecule is required to engage a differing type of said specific cell-surface receptor in order for said configurable medical treatment payload delivery means to insert said quantity of nuclear signaling proteins into intended said predetermined biologically active cells of a particular type.

17. The configurable medical treatment payload delivery means in claim 11 wherein said external probes are comprised of a quantity of glycoprotein molecules.

18. The glycoprotein molecules in claim 17 wherein said glycoprotein molecules are comprised of a protein segment which extends outward and away from said exterior envelope, which is attached to a carbohydrate segment, said carbohydrate segment intended to be embedded in said exterior envelope to hold said glycoprotein molecule affixed to said exterior envelope,
whereby said protein segment which extends outward and away from said exterior envelope is intended to engage said specific cell-surface receptor on said predetermined biologically active cells of a particular type.

19. The glycoprotein molecules in claim 17 wherein said glycoprotein molecules are comprised of a plurality of glycoprotein molecules,
whereby, at least two differing configurations of said glycoprotein molecules may be needed to successfully engage said predetermined biologically active cells of a particular type with one type of configuration of said glycoprotein molecule engaging a type of said specific cell-surface receptor, while a differing type of configuration of said glycoprotein molecule is required to engage a differing type of said specific cell-surface receptor in order for said configurable medical treatment payload delivery means to insert said quantity of nuclear signaling proteins into said predetermined biologically active cells of a particular type.

20. The nuclear signaling proteins in claim 11 wherein said nuclear signaling proteins are selected from the group consisting of nuclear receptors, nuclear binding proteins, and artificial transcription factors.
